(19) United States
(12) Patent Application Publication
(10) Pub. No.: US 2011/0180445 A1
(43) Pub. Date: Jul. 28, 2011

(54) Cosmetic tablets and capsules for direct delivery of active ingredients

(76) Inventor: Marni Markell Hurwitz, Far Hills, NJ (US)

(21) Appl. No.: 13/066,028

(22) Filed: Apr. 5, 2011

Related U.S. Application Data
(63) Continuation-in-part of application No. 12/156,609, filed on Jun. 3, 2008.

(51) Int. Cl. B65D 73/00; A61K 8/11; A61K 8/89; A61K 8/92; A61K 8/65; A61K 8/73; A61Q 1/02; A61Q 1/06; A61Q 19/10; A61Q 1/12; A61Q 19/00; A61Q 13/00

(52) U.S. Cl. 206/461; 424/401; 424/78.03; 510/137; 510/138; 510/141; 424/59; 424/63; 424/64; 424/69; 424/43; 512/4

(57) ABSTRACT

A cosmetic tablet or capsule for direct delivery of active ingredients onto a keratinous substrate is provided composed of a tablet or capsule having an outer shell and inner cavity containing at least one active ingredient optionally in a physiologically acceptable carrier. Active ingredients contained and delivered by the cosmetic tablet include fragrances, perfumes, serums, lotions, creams, sunscreens, moisturizers, body wash, shower gels, foundations and a plethora of products for making-up or applying to the skin. Other ingredients, such as scents, dyes, adjuvants, moisturizers, and/or colorings may be included in the composition and hosed in the tablet or capsule. In one embodiment the outer shell of the tablet/capsule is substantially water soluble and the active ingredients are appointed to be released by dissolving or puncturing the outer shell. In another embodiment the outer shell of the table/capsule is substantially non-water soluble and the active ingredients is released by puncturing the outer shell.

### BACKGROUND OF THE INVENTION

1. Field of the Invention

The present invention relates to cosmetic tablets and capsules appointed to house and directly release active ingredients for direct application onto a keratinous substrate and method of use; and, more particularly, to cosmetic tablets and capsules containing active ingredients therein associated with cosmetics and toiletries including, inter alia, fragrances, perfumes, serums, lotions, creams, moisturizers, body wash, shower gels, foundations and a plethora of products for making-up or applying to the skin.

2. Description of the Prior Art

Travel is becoming increasingly difficult with restrictions being placed on airline bag weight, bag check number limitations, and issues concerning liquids located in canyon baggage. Currently available cosmetic products are sold in ranging sized containers, extending from large bottles or tubes to smaller trial or travel size bottles and tubes. When traveling, large tubes and bottles can be quite cumbersome, taking up far too much space in one's luggage, carryon or purse. Thus, large bottles and containers or tubes of cosmetic products prove to be quite cumbersome. Even still, the smaller bottles or tubes can be a nuisance as they generally only hold a small amount and are exhausted relatively quickly in the course of a vacation or day, requiring one to pack several small bottles or tubes.

Further still, many perfumes and cosmetic products are desired to be carried in one's purse daily in order for reapplication throughout the day or evening. Additionally, many perfumes, soaps and cosmetic products are desired to be sampled in small trial sizes in order for consumers to determine whether to purchase the product. The same disadvantageous result as set forth above: large bottles or tubes are not convenient to carry on one's purse and are not cost advantageous as samples, and smaller bottles and tubes are exhausted readily and require a number of small bottles/tubes to be carried in one's purse or handed out as samples. Further, costs associated with the current packaging can become expensive over time.

The vast majority of perfumes, soaps and cosmetic products provide various types of packaging, generally composed of a polymeric material in the form of a container appointed for multiple servings therefrom. These types of large containers include measured quantity dispenser devices, such as that disclosed by U.S. Pat. No. 6,244,470 to Harley-Wilmot; U.S. Pat. No. 6,805,262 to Frazier; and U.S. Pat. No. 6,814,261 to Gebrayel. These larger containers with multiple servings therein are too cumbersome to pack or carry when traveling or the like, and they do not provide capsule delivery directly.

One-time use packages of mouthwash have been provided, generally in the form of a small container with a single use measured amount of mouthwash therein, that is dispensed from the container, including that disclosed by U.S. Pat. No. 5,002,189 to Sahi and U.S. Pat. No. 4,865,481 to Scales. Although appointed for single use, these devices do not provide a capsule with perfumes, soaps and cosmetic products therein for direct delivery onto a keratinous substrate/skin.

Various mouthwash tablets have been provided, the tablets do not provide a direct delivery of perfumes, soaps and cosmetic products, but instead provide tablets or powder with mouthwash, etc., many of which are appointed to be mixed with water or other ingredients to form a mouthwash at the time of use. Including that disclosed by: U.S. Pat. Nos. 3,772,431 and 3,888,976 to Mlkvy, et al.; and U.S. Patent App. Publ. 20020114767 to Rolla. Likewise, many patents address issues related to providing nutritional supplements in a powder form, pill form or mixture appointed for addition to a beverage. Foe examples: U.S. Pat. Nos. 5,032,411, 5,114,723 to Stray-Gundersen; U.S. Pat. No. 5,609,897 to Chandler, et al.; U.S. Pat. No. 5,681,606 to Hutchison, et al.; U.S. Pat. No. 6,287,596 to Murakami, et al., U.S. Pat. No. 5,332,096 to Battaglia. However, these various tablets and nutritional supplements do not provide direct delivery of perfumes, soaps and cosmetic products by way of a tablet or capsule.

Moreover, various toothpaste chewing gums and tablets forming a past upon chewing have been provided. For example, see U.S. Pat. No. 4,259,355 to Marmo, et al.; and U.S. Pat. No. 5,057,305 to Aberg. These gums and chewing tablets are not are not provided as encapsulated perfumes, soaps and cosmetic products.

There remains a need in the art for a cosmetic capsule and tablet appointed to release active ingredients directly onto a person's keratinous substrate and method of application thereof. More particularly, there is a need in the art for cosmetic tablets and capsules containing active ingredients therein associated with cosmetics and toiletries including, inter alia, fragrances, perfumes, serums, lotions, creams, moisturizers, body wash, shower gels, foundations and a plethora of products for making-up or applying to the skin.

### SUMMARY OF THE INVENTION

The present invention is directed to a cosmetic capsule and/or tablet appointed to release active ingredients directly onto a person's keratinous substrate/skin and method of application thereof. More particularly, the present invention is directed to tablets and capsules containing active ingredients therein associated with cosmetics and toiletries including, inter alia, fragrances, perfumes, serums, lotions, creams, moisturizers, body wash, shower gels, foundations and a plethora of products for making-up or applying to the skin.

In one embodiment, the cosmetic tablet or capsule composed of a substantially water soluble material, such as a gelatin, gelatin mixture, organogel, starch, or other capsule ingredients, to encapsulate the active ingredients therein. In this embodiment, the cosmetic table or capsule is appointed to be capable of dissolving in water. Particular applications concerning the dissolvable capsule/table version include wherein the cosmetic is a shower gel, soap, or face wash product. In another embodiment, the cosmetic tablet includes an outer shell composed of a pressed powder with binders and includes effervescent properties so that it readily dissolves in an effervescent effect, dispersing the ingredients on the user's skin in the shower, bath or sink. Preferably, in these embodiments, the capsule or tablet rapidly dissolves in water to deliver the active ingredients.

In another embodiment, the cosmetic tablet or capsule may be composed of a thin polymeric or silicone film that is not water soluble and is instead appointed to be punctured at the time of use to release the cosmetic product. Examples of cosmetic products contemplated include foundations, blushes, lipsticks, eye shadows, lotions, serums, creams, or moisturizers.

The tablet or capsule includes active ingredients housed within an inner cavity protected by an outer shell. These active ingredients comprise: cosmetics and toiletries including, inter alia, fragrances, perfumes, serums, lotions, creams, moisturizers, body wash, shower gels, foundations and a plethora of products for making-up or applying to the skin. Other ingredients, such as fragrances, scents, colorings and dyes may be included in the composition of the tablet or capsule. Delivery of the cosmetic ingredients is effectuated by simply dissolving the capsule/tablet in water to cause the outer shell to dissolve in one embodiment, or puncturing the capsule/tablet in another embodiment and allowing the cosmetic product to be released.

In one embodiment, a cosmetic capsule/tablet for direct delivery of at least one active ingredient onto a keratinous substrate is provided including an outer shell and an inner cavity. The outer shell is appointed to dissolve or break down in water or is appointed to be punctured by a user. The active ingredient is contained within the inner cavity of the capsule/tablet. The outer shell is appointed to dissolve or break depending on its composition to directly deliver the active ingredients to the user during application.

The outer shell of the capsule/tablet may be composed of a substantially water soluble material so that it dissolves in water, including: effervescent ingredients comprising a mixture of citric acid and sodium bicarbonate as in a compressed powder form with binders, a gelatin composition to microencapsulate the active ingredients, a combination of gelatin and glycerin, a coating agent, a plant-based gelling substances like carrageenans and modified forms of starch and cellulose, an organogel, and/or a gum compound. Preferably, the tablet is sold or dispensed in a peel away package with a plurality of individual tablet compartments for individually housing the tablets therein, and has particular applications in compact storage and as travel tablets.

Alternatively, the outer shell of the capsule/tablet may be composed of a substantially non-water soluble material. In this embodiment the outer shell is appointed to be discarded after it is opened and manipulated to release and directly deliver the active ingredients onto the skin of a user.

Further, a cosmetic capsule/tablet system for direct delivery of at least one active ingredient onto a keratinous substrate is provided. The system comprises: (a) a package including one or more peel away compartments each housing an tablet or capsule therein; and (b) the tablet or capsule comprising: (i) an outer shell and an inner cavity, wherein the outer shell is appointed to dissolve or break down in water or is appointed to be punctured by a user; and (ii) the active ingredient is contained within the inner cavity of the capsule/tablet. Whereby the outer shell dissolves or breaks to directly deliver the active ingredients to the user during application.

### BRIEF DESCRIPTION OF DRAWINGS

The invention will be more fully understood and further advantages will become apparent when reference is had to the following detailed description and the accompanying drawings, in which:

FIG. 1 is a schematic view of a peel away package with a plurality of individual tablet compartments individually housing cosmetic capsules/tablets therein, wherein the peel away package comprises tab portions for readily accessing each of the individual tablet compartments and a cosmetic tablet is shown removed from one of the compartment and ready for use;

FIG. 2 is a cross-sectional view of the package of FIG. 1, taken along planer line V;

FIG. 3 is a cross-sectional view of the cosmetic tablet or capsule of FIG. 1, taken along planer line X;

FIG. 4 is a cross-sectional view of the cosmetic tablet or capsule of FIG. 1, taken along planer line X, along with a view of an optional pin or puncture device; and

FIG. 5 is a schematic view of a container of cosmetic tablets and/or capsules wherein the outer shell (and visa vie the tablet and/or capsule) is shaped in a variety of shapes and sizes.

### DESCRIPTION OF THE PREFERRED EMBODIMENTS

The present invention is directed to a cosmetic tablet or capsule for direct delivery of active ingredients onto a keratinous substrate is provided composed of a tablet or capsule having an outer shell and inner cavity containing at least one active ingredient optionally in a physiologically acceptable carrier. Active ingredients contained and delivered by the cosmetic tablet include fragrances, perfumes, serums, lotions, creams, sunscreens, moisturizers, body wash, shower gels, foundations and a plethora of products for making-up or applying to the skin. Other ingredients, such as scents, dyes, adjuvants, moisturizers, and/or colorings may be included in the composition and hosed in the tablet or capsule. In one embodiment the outer shell of the tablet/capsule is substantially water soluble and the active ingredients are appointed to be released by dissolving or puncturing the outer shell. In another embodiment the outer shell of the table/capsule is substantially non-water soluble and the active ingredients is released by puncturing the outer shell.

More particularly, the present invention is directed to tablets and capsules containing active ingredients therein associated with cosmetics and toiletries including, inter alia, fragrances, perfumes, serums, lotions, creams, moisturizers, body wash, shower gels, foundations and a plethora of products for making-up or applying to the skin.

In one embodiment, the cosmetic tablet or capsule composed of a substantially water soluble material, such as a gelatin, gelatin mixture, organogel, starch, or other capsule ingredients, to encapsulate the active ingredients therein. In this embodiment, the cosmetic table or capsule is appointed to be capable of dissolving in water. Particular applications concerning the dissolvable capsule/table version include wherein the cosmetic is a shower gel, soap, or face wash product. In another embodiment, the cosmetic hygiene tablet includes an outer shell composed of a pressed powder with binders and includes effervescent properties so that it readily dissolves in an effervescent effect, dispersing the ingredients on the user's skin in the shower, bath or sink. Preferably, in these embodiments, the capsule or tablet rapidly dissolves in water to deliver the active ingredients.

In another embodiment, the cosmetic tablet or capsule may be composed of a thin polymer or silicone film that is not water soluble and is instead appointed to be punctured at the time of use to release the cosmetic product. Examples of cosmetic products contemplated include foundations, blushes, lipsticks, eye shadows, lotions, serums, creams, or moisturizers.

The tablet or capsule includes active ingredients housed within an inner cavity protected by an outer shell. These active ingredients comprise: cosmetics and toiletries including, inter alia, fragrances, perfumes, serums, lotions, creams, moisturizers, body wash, shower gels, foundations and a plethora of products for making-up or applying to the skin. Other ingredients, such as fragrances, scents, colorings and dyes may be included in the composition of the tablet or capsule. Delivery of the cosmetic ingredients is effectuated by simply dissolving the capsule/tablet in water to cause the outer shell to dissolve in one embodiment, or puncturing the capsule/tablet in another embodiment and allowing the cosmetic product to be released.

Surprisingly and unexpectedly, there are several advantages obtained by including cosmetic active ingredients within a tablet or capsule. Such cosmetic tablets provide a unique, novel system for safe carriage of active ingredients, such as perfumes, fragrances, make-up, lotions, sunblock, moisturizers, soaps and body washes, during travel, while also providing compact storage. The active ingredient capsules/tablets herein address problems prevalent in the travel industry. In the modern day travel environment carried-on items are seriously evaluated and liquids can only be of a predetermined quantity, otherwise they are discarded by security. Sizing means so much when traveling with toiletries. The cosmetic tablet uniquely addresses this recurring, ever increasing problem, and helps to eliminate stress associated with traveling.

Moreover, in today's hustle and bustle society, compacting everyday items and electronics is becoming increasingly important to the consumer. The cosmetic/beauty tablets provide consumer the ability to purchase multiple packages of tablets having various cosmetic active ingredients therein, in a compact unique way that allows for compact storage and easy carrying. For example, where the cosmetic tablet herein is a perfume tablet, the user simply can carry the cosmetic tablets to work, or a restaurant, so that the user can readily apply new perfume in the evening, without the necessity of dealing with a large bottle of perfume which can be difficult to carry in a clutch or small purse. Moreover, the tablets can be presented in combinations—so that a package of tablets include equal number of foundation tablets and lipstick/lip gloss tablets so that the user can reapply make-up when on the go.

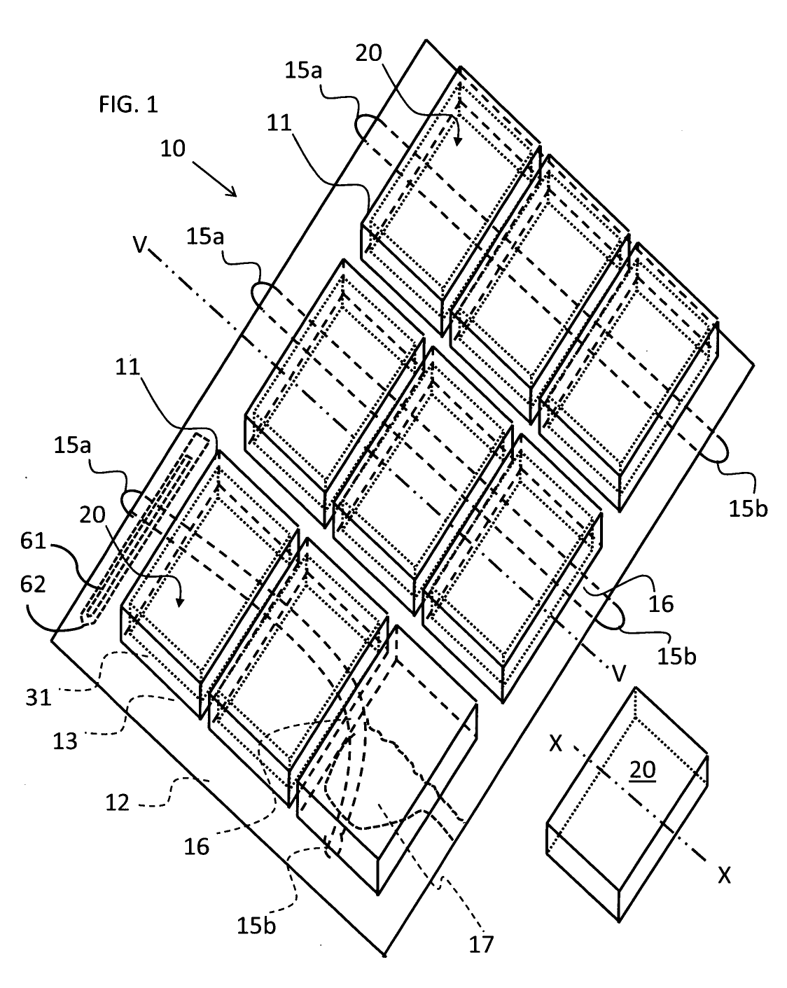

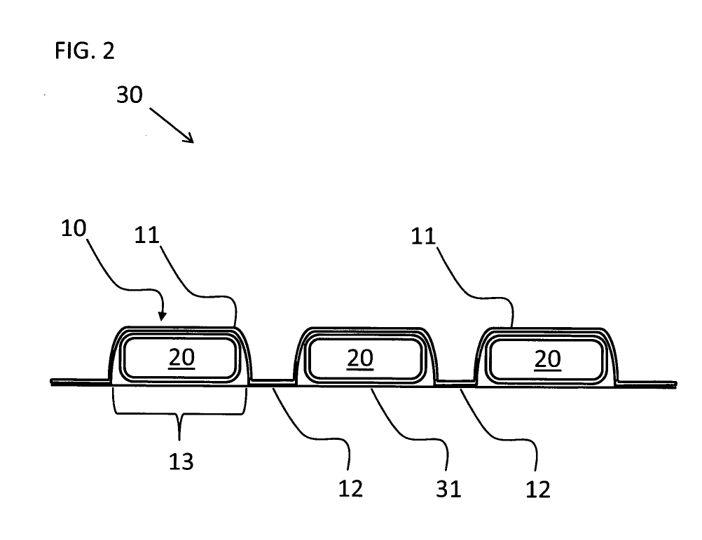

FIG. 1 is a schematic view of a peel away package with a plurality of individual tablet compartments housing cosmetic tablets therein, shown generally at 10. FIG. 2 is a cross-sectional view of the package of FIG. 1, taken along planer line V, shown generally at 30. In referring to FIGS. 1 and 2, package 10 includes one or more peel away compartments 11 each housing a cosmetic tablet/capsule 20 therein. Package 10 is generally comprised as a flexible, polymeric package with compartments 11 molded therein, with a flat under surface 12, and compartment apertures 13 for each compartment 11. A foil 31, or other covering, extends over flat under surface 12 of package 10, extending over each compartment aperture 13 to secure cosmetic hygiene tablet 20 within compartment 11 during storage and before use. In the embodiment shown, package 10 comprises tab portions 15a, 15b with strip 16 therebetween. As shown, when tab portion 15b (or 15a) is pulled, strip 16 cuts through foil 31 to form an opening 17 which is further torn by the user for accessing compartment 11 and cosmetic tablet 20 therein for removal of tablet 20. An optional pin or puncture device 62 is shown housed in a puncture device compartment 61.

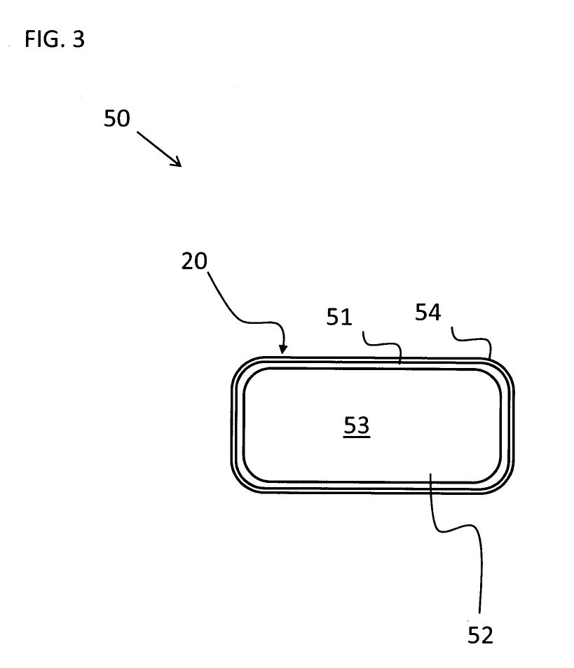

FIG. 3 is a cross-sectional view of the cosmetic tablet or capsule of FIG. 1, taken along planer line X, shown generally at 50. Cosmetic tablet 20 or capsule comprises an outer shell 51 and an inner cavity 52 containing active ingredients 53. Outer shell 51 preferably further comprises a coating agent 54, including carnauba wax to yield a glossy finish and enhanced protection of outer shell 51 from moisture during storage.

In a first embodiment, outer shell 51 is composed of a substantially water soluble material and is appointed to rapidly dissolve in water to deliver active ingredients 53 to the skin of the user, such as during a shower or bath, or in a sink when washing one's face or hands. Outer shell 51 is appointed to dissolve or break down in water at the time of application of the active ingredients. Outer shell 51 (and coating agent 54) is preferably very thin, just providing enough coverage to protect and/or encapsulate active ingredients 53 therein. Active ingredients 53 contained within inner cavity 52 of tablet 20 are released as outer shell 51 dissolves or is penetrated, and directly infuses the skin to deliver active ingredients 53 to the skin. Particular applications concerning the dissolvable outer shell 51 include active ingredients 53 including a shower gel, soap, hand wash or face wash product. In another embodiment, the outer shell 51 is composed of a pressed powder with binders and includes effervescent properties so that it readily dissolves in an effervescent effect, dispersing the ingredients onto the user's skin in the shower, bath or sink. Preferably, in these embodiments, the capsule or tablet rapidly dissolves in water to deliver the active ingredients. The outer shell 51 thusly may be composed of effervescent ingredients comprising a mixture of citric acid and sodium bicarbonate. Alternatively, the outer shell 51 is composed of a gelatin, glycerin, plant-based gelling substances like carrageenans and modified forms of starch and cellulose, or an ogranogel, or mixtures thereof. Further, the outer shell 51 may include a coating agent, including carnauba wax, sugars, or other thin films.

In another embodiment, outer shell 51 is composed of a thin polymeric or silicone film that is not water soluble and is instead appointed to be punctured at the time of use to release the cosmetic product. Examples of cosmetic products contemplated include foundations, blushes, lipsticks, eye shadows, lotions, serums, creams, or moisturizers and/or oil based cosmetic products that would dissolve an otherwise water soluble shell.

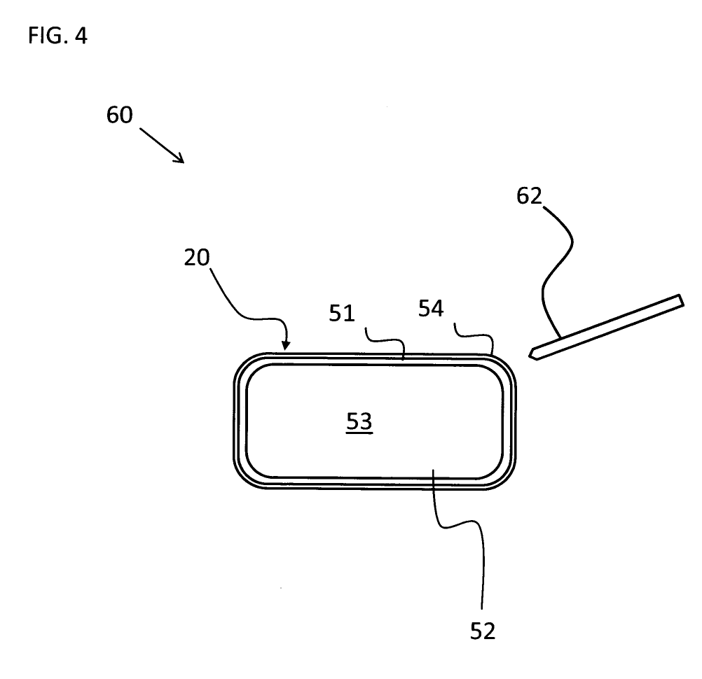

FIG. 4 is a cross-sectional view of the cosmetic tablet or capsule of FIG. 1, taken along planer line X, along with a view of an optional pin or puncture device, shown generally at 60. In this manner, the pin or puncture device 62 is appointed to be utilized to puncture or break the outer shell 51 of the tablet 20. The puncture device 62 may be composed of a rigid polymeric material or a metal or aluminum material and generally has a pointed tip for penetrating the outer shell of the tablet 20.

The active ingredient is optionally carried in a physiologically acceptable carrier medium, in turn housed within tablet 20. The carrier medium may be any cosmetically acceptable medium for carrying active ingredients in cosmetic and beauty products, including chosen from aqueous, oils, emulsions (w/o; o/w; etc.), silicones (Si/w; w/Si; etc.) and/or combinations thereof. Excipients, or inactive ingredients, may be included, such as water soluble binders; fragrances; scents; pigments to make tablets visually attractive; and effervescent enabling ingredients (such as citric acid and sodium bicarbonate). A soluble coating may be applied to provide aesthetics, or to make it more resistant to the environment, extending its shelf life. A wide variety of binders may be used, including starch, cellulose, lactose powder, dibasic calcium phosphate, sucrose, corn (maize) starch, microcrystalline cellulose and modified cellulose (for example hydroxymethyl cellulose), and/or sorbital.

In one embodiment, outer shell 51 is composed of effervescent ingredients comprising a mixture of citric acid and sodium bicarbonate so that outer shell 51 rapidly effervescently dissolves in water to release the active ingredients 53. In this manner, active ingredients 53 preferably are a paste or powder rather than a liquid. Ingredients utilized to form an effervescent outer shell 51 include preferably a mixture of citric acid and sodium bicarbonate, which, when exposed to water undergo chemical reaction to yield carbon dioxide and sodium citrate. Powdery ingredients may be pulverized to a fine mesh particle size to enable compression into tablet form, while having large enough surface area for quick dissolution when in water/when dropped in the liquid. For example 100 mesh particles have an average diameter of 140 microns while a 325 mesh particles have a diameter of only 44 microns thereby presenting a larger surface area. A 5 micron particle size results in a significantly larger surface area available for dissolution. Liquid or sticky ingredients may be microencapsulated in the effervescent tablets, which are free flowing or readily dissolving.

Wherein tablet 20 is a capsule type delivery method, outer shell 51 may be composed of a water soluble material (i.e. a gelatin composition; starch; etc.) or of a substantially non-soluble material (i.e. polymer; silicone). More preferably, wherein the outer shell 51 is soluble, it is composed of a combination of gelatin and glycerin (2% or less), mineral oil (2% or less). Outer shell 51 may comprise a coating agent, including carnauba wax to provide a glossy finish and enhance protection of outer shell 51 from the environment. A plant-based gelling substance like carrageenans and modified forms of starch and cellulose may be used to form outer shell 51 in forming the capsule 20. An organogel may be utilized forming outer shell 51, comprising non-crystalline, non-glassy thermoreversible solid materials composed of a liquid organic, such as an organic solvent, a mineral oil or a vegetable oil. The solubility and particle dimensions of the structurant are important characteristics for the elastic properties and firmness of the organogel. Preferably, the capsule is a water quick-dissolving seamless capsule having thin walls. Such as that indicated by U.S. Pat. No. 7,025,911; or through various capsule preparation techniques. See publication “Microencapsulation” by Herbig as presented in the “Encyclopedia of Chemical Technology” of Kirk-Othmer, Volume 13, 2nd Edition, pp 436-456. Optionally, outer shell 51 is composed of a gum compound. Outer shell 51 may be soft or brittle so that it can be broken into smaller particles for more rapid dissolution in the water. In another embodiment, outer shell 51 is not soluble and therefore is not dissolved in the water, but instead is a polymeric material (thin and flexible) that is broken, cut or penetrated to release active ingredients in liquid form, which are poured onto the skin and outer shell 51 discarded into a trash receptacle.

Active ingredient 53 may be a body wash, shower gel, soap, hand soap and/or face soap, a perfume and/or fragrance, a serum, lotion, cream, sun block, and/or moisturizer, a make-up product chosen from foundation, blush, concealer, eye shadow and/or lipstick, and/or combinations thereof. The active ingredient 53 may be in liquid form, gel, paste, and or powder form. A variety of auxiliary ingredients may be added in the active ingredient composition, including for example, adjuvants, conditioners, vitamins, essential oils, self-tanners, tints, dyes, fragrances, sunscreen actives (organic and/or inorganic). Further, the active ingredient comprises at least one medical agent, such as for example retinal or wrinkle agents, or acne agents.

Preferably the active ingredient (with or without the carrier) is provided in an amount suitable for single use application to a user's skin, preferably ranging from 0.25 to 0.5 ounces. Wherein the active ingredient 53 is a liquid, the outer shell of tablet 20 is formed as a capsule to contain the liquid and prevent leakage. Composition of the capsule is that as discussed hereinabove, in utilizing common capsule manufacturing processes. In order to prevent dissolution of outer shell 51 when the outer shell is composed of a water soluble material during storage and before use, preferably the active ingredient/carrier is an essential oil based composition rather than a water or alcohol based composition as the alcohol would cause the capsule to degrade and dissolve. Preferably the active ingredient/carrier is essential oil. Other active ingredients, such as herbal ingredients for providing a soothing effect to the skin, such as menthol, extract of piper cubeba, glycyrrhiza glabra, acorus calamus alpinia galanga, aloe vera gel, goldenseal, calendula, blood root, grapefruit seed extract, flavors (natural), citric acid, echinacea, chamomile, and vitamin b12. Such herbal ingredients, particularly aloe vera gel, goldenseal, calendula and blood root aid in protecting tissue, while soothing same from irritants and temporarily relieving sore throat pain or discomfort. Numbing agents, such as clove oil (small amount), chamomilla 3×HPUS, benozocaine (7.5% or less), belladonna 3×HPUS (0.0003% Alkaloids)—for redness and inflammation, may be utilized to aid in providing comfort to the gums, cosmetic tissue and/or teeth of the user.

The active ingredient may also contain agents for the artificial tanning and/or browning of the skin (self-tanning agents) such as, for example, dihydroxyacetone (DHA). The cosmetic active ingredient according to the invention may also contain pigments or, alternatively, nanopigments (average size of the primary particles: generally ranging from 5 nm to 100 nm, preferably from 10 to 50 nm) of coated or uncoated metal oxides such as, for example, nanopigments of titanium dioxide (amorphous or crystallized in rutile and/or anatase form), of iron oxide, of zinc oxide, of zirconium oxide or of cerium oxide, which are all photoprotective agents that are per se well known to this art and which are effective by physical blocking (reflection and/or diffusion) of the UV irradiation. Conventional coating agents include, moreover, alumina and/or aluminum stearate.

The compositions of the invention may additionally comprise conventional cosmetic additives and adjuvants selected especially from among fats, organic solvents, ionic or nonionic thickening agents, softeners, antioxidants and especially anti-free-radical antioxidants, opacifying agents, stabilizing agents, emollients, silicones, a-hydroxy acids, anti-foaming agents, hydrating agents, vitamins, fragrances, preservatives, surfactants, fillers, insect repellants, sequestering agents, polymers, propellants, basifying or acidifying agents, dyes and colorants, or any other ingredient usually employed in cosmetics, in particular for the production of sunscreen/cosmetic compositions in emulsion form.

The fats may comprise an oil or a wax or mixtures thereof, and may also comprise fatty acids, fatty alcohols and fatty acid esters. The oils may be selected from among animal, plant, mineral or synthetic oils and, especially, from among liquid petrolatum, paraffin oil, volatile or non-volatile silicone oils, isoparaffins, poly-α-olefins, fluoro oils and perfluoro oils. Similarly, the waxes may be selected from among animal, fossil, plant, mineral or synthetic waxes that are per se known to this art. Exemplary organic solvents include the lower polyols and alcohols. The thickening agents may be selected, especially, from among crosslinked polyacrylic acids, modified or unmodified guar gums and celluloses such as hydroxypropyl guar gum, methylhydroxyethylcellulose and hydroxypropylmethylcellulose.

The compositions carrying the active ingredients of the invention may be formulated according to techniques well known to this art, in particular those intended for the preparation of emulsions of oil-in-water, water-in-oil type, silicone-in-water or water-in-silicone type.

Tablet/capsule 20 may further comprise vitamin compositions and/or mineral compositions. Alternatively, the nutritional ingredient may be an electrolyte composition. Preferably, the vitamin composition includes vitamin A, vitamin B1, B2, B6, B12, vitamin C, vitamin E and coenzyme (CQ10). Typical vitamins in powder form includes vitamin C, B1, B2, B6, B12, folic acid coenzyme (CQ10) and D. Typical liquid or semi-solid vitamins include vitamin A and E. Mineral compositions may include calcium compounds, preferably calcium citrate, magnesium compound, preferably magnesium citrate, chromium compound, preferably chromium picolinate or Niacin chromium GTF complex, zinc compound, selenium compound, iron compounds and others. Anti-oxidants include coenzymes. Hydrating substances include sodium and potassium chloride. Fragrance enhancing ingredients preferably include orange flavor, lemon or lime flavor, guava flavor, mango flavor, dragon fruit flavor, green tea, caffeine and guarana, peach flavor, or strawberry flavor.

The formulation may contain inactive ingredients such as microcrystalline cellulose, croscamellose sodium, silica, magnesium stearate, pharmaceutical glaze and other ingredients that improve processability of the composition and the texture of the final product.

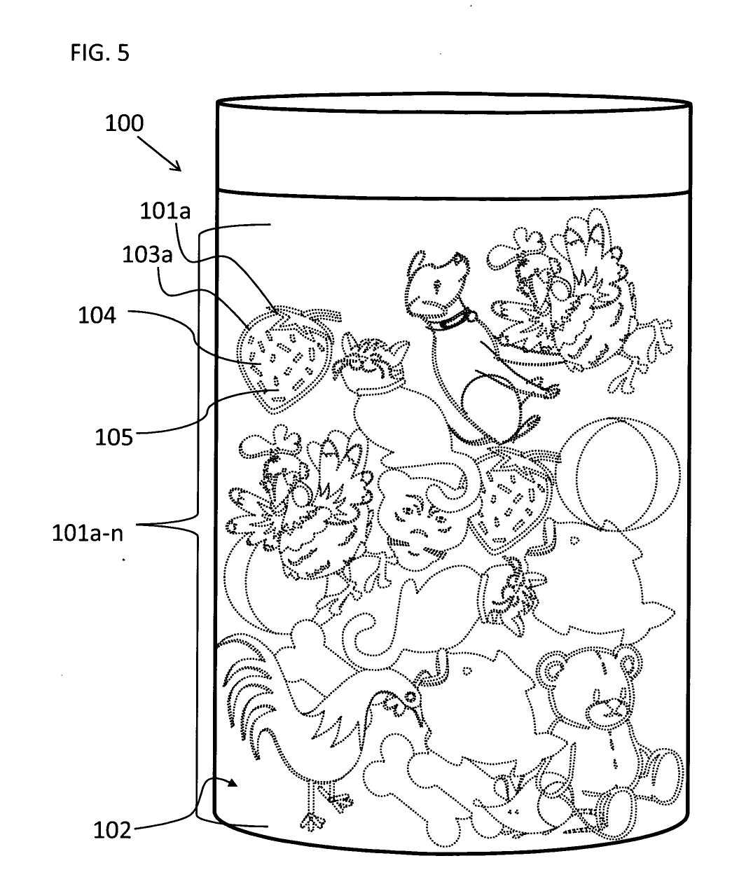

FIG. 5 is a schematic view of a container of cosmetic tablets and/or capsules wherein the outer shell (and visa vie the tablet and/or capsule) is shaped in a variety of shapes and sizes, shown generally at 100. Cosmetic tablets 101 (101a-n) are shown housed in a container 102, and are configured so that the outer shell 103 of tablets 101 are of a plethora of shapes comprising a shape in form of an animal, creature, toy, object or character (specifically pointing to 101a, 103a as a representative shaped tablet 101). As constructed, cosmetic tablet 101 include outer shell 103 with an inner cavity 104 containing active ingredients 105 therein, as shown by way of tablet 101a. The cosmetic tablets and capsules may be of a plethora of shapes, including parallelograms in general, circles, ovals, and combinations of same. Further, these cosmetic tablets and capsules may be formed in the shape of animals or creatures, toys, and other objects to yield imaginary animal and object shaped cosmetic hygiene tablets and capsules, as shown. These imaginary animals and objects would be appealing to women, men, teenagers and children, and would encourage use of the cosmetic tablets and capsules at school, camp, trips, college and the like. The animal and object shaped cosmetic hygiene tablets and capsules could be packaged in the manner previously described (i.e. in a packet having discrete compartments therein as shown in the aforediscussed FIGS. 1 and 2), or stored in a bottle or like vessel so that the creatures and imaginary objects would be visible to young people, thereby encouraging usage, improving beauty and proper hygiene.

Continuing with FIG. 5, preferably, tablets 101a-n may be of different shapes, sizes and/or colors—and may be demarcated via color, for example, to illustrate different active ingredient 105 characteristics within a specifically colors/shaped tablet 101a-n so that container 102 may contain a variety of tablets 101a-n having a variety of different active ingredients 105a-n therein. For example, one color tablet 101a may be a perfume; another tablet shape/color may be a lotion for daytime having sunblock and skin tinting therein; another tablet shape/color may denote a nighttime moisturizing lotion; another tablet shape/color may denote an astringent or toner; another may denote a foundation; etc. Different shapes/colors may be part of a beauty or face care regime, wherein one tablet shape or color goes to a face wash, another to a toner, another to a daytime lotion, another to a night time serum and/or lotion. Shapes shown in container 102 via tablets 101a-n are merely representative, but a plethora of different shapes may be provided. The animal shaped tablets 101a-n can be well known characters or trademarks to aid in attracting consumers while providing a marketing tool for companies owning the character or/and trademark or brand image. Container 102 may be a typical container, box, cartridge, decorative dispenser, or other type of container, such as that discussed in reference to FIGS. 1 and 2.

Having thus described the invention in rather full detail, it will be understood that such detail need not be strictly adhered to, but that additional changes and modifications may suggest themselves to one skilled in the art, all falling within the scope of the invention as defined by the subjoined claims.

What is claimed is:

1. A cosmetic capsule/tablet for direct delivery of at least one active ingredient onto a keratinous substrate, comprising:
a. an outer shell and an inner cavity, wherein said outer shell is appointed to dissolve or break down in water or is appointed to be punctured by a user; and
b. said active ingredient is contained within said inner cavity of said capsule/tablet;
whereby said outer shell dissolves or breaks to directly deliver said active ingredients to said user during application.

2. A cosmetic capsule/tablet as recited by claim 1 comprising a physiologically acceptable carrier medium carrying said active ingredient housed in said inner cavity of said capule/tablet.

3. A cosmetic capsule/tablet as recited by claim 2, wherein said carrier medium is any cosmetically acceptable medium for carrying active ingredients in cosmetic and beauty products, including chosen from aqueous, oils, emulsions, silicones and/or combinations thereof.

4. A cosmetic capsule/tablet as recited by claim 1, wherein said outer shell is of a shape comprising a shape in form of an animal, creature, toy, object or character.

5. A cosmetic capsule/tablet as recited by claim 1, wherein and said outer shell is substantially water soluble.

6. A cosmetic capsule/tablet as recited by claim 5, wherein and said outer shell is composed of a gel-like substance that substantially dissolves in water to release said product onto said keratinous substrate.

7. A cosmetic capsule/tablet as recited by claim 1, wherein said active ingredient is a body wash, shower gel, soap, hand soap and/or face soap.

8. A cosmetic capsule/tablet as recited by claim 1, wherein said active ingredient is a perfume and/or fragrance.

9. A cosmetic capsule/tablet as recited by claim 1, wherein said active ingredient is a serum, lotion, cream, sun block, and/or moisturizer.

10. A cosmetic capsule/tablet as recited by claim 1, wherein said active ingredient is a make-up product chosen from foundation, blush, concealer, eye shadow and/or lipstick.

11. A cosmetic capsule/tablet as recited by claim 1, wherein said active ingredient is in substantially liquid form.

12. A cosmetic capsule/tablet as recited by claim 1, wherein said active ingredient is in substantially powder form.

13. A cosmetic capsule/tablet as recited by claim 1 comprising auxiliary ingredients chosen from adjuvents, conditioners, vitamins, essential oils, self-tanners, tints, dyes, and fragrances.

14. A cosmetic capsule/tablet as recited by claim 1, wherein said active ingredients comprise sunscreen actives.

15. A cosmetic capsule/tablet as recited by claim 1, wherein said active ingredient comprises at least one medically effective agent.

16. A cosmetic capsule/tablet as recited by claim 1, wherein said outer shell is composed of effervescent ingredients comprising a mixture of citric acid and sodium bicarbonate.

17. A cosmetic capsule/tablet as recited by claim 1, wherein said outer shell is composed of a gelatin composition.

18. A cosmetic capsule/tablet as recited by claim 1, wherein said outer shell is composed of a combination of gelatin and glycerin.

19. A cosmetic capsule/tablet as recited by claim 1, wherein said outer shell comprises a coating agent, including carnauba wax.

20. A cosmetic capsule/tablet as recited by claim 1, wherein said outer shell is composed of plant-based gelling substances like carrageenans and modified forms of starch and cellulose.

21. A cosmetic capsule/tablet as recited by claim 1, wherein said outer shell is composed of an organogel.

22. A cosmetic capsule/tablet as recited by claim 1, wherein said outer shell is not substantially insoluble in water and wherein said outer shell is appointed to be discarded after it is opened and manipulated to release and directly deliver said active ingredients onto said keratinous substrate of said user.

23. A cosmetic capsule/tablet as recited by claim 1, wherein said outer shell is composed of a polymer.

24. A cosmetic capsule/tablet as recited by claim 1, wherein said outer shell is composed of a silicone.

25. A cosmetic capsule/tablet as recited by claim 1 comprising a peel away package with a plurality of individual tablet compartments for individually housing said capsule/tablets therein.

26. A cosmetic capsule/tablet as recited by claim 26, wherein said peel away package comprises tab portions for readily accessing each of said individual tablet compartments.

27. A cosmetic capsule/tablet as recited by claim 1, wherein said active ingredient optionally in a carrier medium in said inner cavity ranges from 0.25 to 0.5 ounces.

28. A cosmetic capsule/tablet system for direct delivery of at least one active ingredient onto a keratinous substrate, comprising:
a. a package including one or more peel away compartments each housing an tablet or capsule therein;
b. said tablet or capsule comprising:
i. an outer shell and an inner cavity, wherein said outer shell is appointed to dissolve or break down in water or is appointed to be punctured by a user; and
ii. said active ingredient is contained within said inner cavity of said capsule/tablet;
whereby said outer shell dissolves or breaks to directly deliver said active ingredients to said user during application.
